(19) United States
(12) Patent Application Publication
(10) Pub. No.: US 2009/0253794 A1
(43) Pub. Date: Oct. 8, 2009

(54) Fibroblast Activator, Collagen Production Promoter, Collagen Contraction Promoter, Hyaluronic Acid Production Promoter, ATP Production Promoter, Melanin Formation Inhibitor, and Agent for External Application to the Skin

(76) Inventors: Jun Tomono, Hyogo (JP); Takahisa Nakai, Hyogo (JP); Toshihide Fujii, Hyogo (JP)

Correspondence Address: WENDEROTH, LIND & PONACK, L.L.P., 1030 15th Street, N.W.,, Suite 400 East, Washington, DC, 20005-1503, US

(21) Appl. No.: 11/989,194

(22) PCT Filed: Jul. 21, 2006
(86) PCT No.: PCT/JP2006/314934

§ 371 Date: Feb. 5, 2008

(30) Foreign Application Priority Data
Jul. 22, 2005 (JP) 2005-212175
Oct. 13, 2005 (JP) 2005-299047
May 29, 2006 (JP) 2006-148622

(51) Int. Cl. A61K 31/195

(52) U.S. Cl. 514/563

(57) ABSTRACT

The present invention is a fibroblast activator, collagen production promoter, collagen contraction promoter, hyaluronic acid production promoter, ATP production promoter or melanin formation suppressor, which contains, as an active ingredient, at least one kind of compound selected from the group consisting of conagenin, a conagenin derivative, and a pharmaceutically acceptable salt thereof, as well as a composition (skin external preparation, cosmetic, pharmaceutical product or food, which is a composition for the improvement of wrinkles or a whitening composition) containing such activator, promoter or melanin formation suppressor.

According to the present invention, a safe skin external preparation, cosmetic (cosmetic composition), pharmaceutical product or food, which shows a high wrinkle improving effect, can be provided, and a safe skin external preparation, cosmetic (cosmetic composition), pharmaceutical product or food, which shows a high whitening effect can be provided.

### TECHNICAL FIELD

The present invention relates to a fibroblast activator, a collagen production promoter, a collagen contraction promoter, a hyaluronic acid production promoter, an ATP production promoter, a melanin formation suppressor and a skin external preparation, as well as cosmetics, which are safe and highly effective.

### BACKGROUND ART

With increasing population of aged people in recent years, prevention of skin aging evidenced by wrinkles, pigmented spot and the like that increases with aging, i.e., so-called antiaging, has been actively studied.

The skin is mainly divided into epidermis, dermis and subcutaneous tissue. Particularly, the fibroblast constituting dermis produces proteins such as collagen and the like and glycosaminoglycans such as hyaluronic acid and the like, forms a binding tissue (extracellular matrix), and plays an important role for the homeostasis of the skin.

Collagen is a protein occupying about ⅓ of the protein in a living body, and is present in a large amount in the blood vessels, skin and bone. Although collagen was once considered a protein with poor nutrition value since it is hardly decomposed by digestive enzymes, since there are reports of enhanced metabolism due to the intake of collagen (JP-A-7-278012), increased diameter of hair (“Nutrition Reports International”, 1976, vol. 13, p. 579), and use as a pharmaceutical agent for the treatment of arthropathy (JP-A-63-39821), its usefulness has been reexamined. Furthermore, since collagen decreases as one grows old, it is considered one cause of angioembrittlement, decreased inelasticity and flexibility of the skin and the like. Particularly, with regard to the skin, it is known that the action of fibroblast becomes weak as one grows old, which weakens the power of the cells to pull collagen fiber (collagen contraction force), resulting in inelasticity of the skin and sagging. Therefore, a highly safe collagen contraction promoter that promotes collagen contraction activity of fibroblast, and eliminates development of skin sagging and loss of skin firmness has been desired.

In recent years, a patent application relating to promoted metabolism of the skin by oral intake of collagen or its hydrolysate has been disclosed (JP-A-7-278012), and a large number of health foods for beauty purposes have been sold. Hyaluronic acid has many functions such as maintenance of water in cellular gap, maintenance of cell tissue based on the formation of jelly-like matrix, maintenance of lubricity and flexibility of tissue, resistance to external force such as mechanical disorder and the like, prevention of bacterial infection, and the like (“BIO INDUSTRY”, 1991, vol. 8, p. 346). For example, it is said that hyaluronic acid in the skin decreases as one grows old, as a result of which aging occurs such as fine wrinkles, drying and the like. While many cosmetics containing, as an aged skin-improving agent, collagen or hyaluronic acid have been proposed, they merely improve surface-moisturizing effect, and do not essentially improve the aging skin. Besides those, vitamins and crude drugs are used as skin cell activators. As the situation stands, however, the treatment of aging skin has not been accomplished yet. Given such situation, attempts have been made to improve aging skin by enhancing collagen production and hyaluronic acid production by the cell itself.

On the other hand, such aging symptoms of human skin, particularly wrinkles and sagging, are also considered to mainly occur due to the degraded function of skin fibroblast and insufficient secretion of matrix fiber and collagen due to degraded function of the cells. Accordingly, activation of skin fibroblast is also considered to be an effective means for the prevention of aging of the human skin or functional improvement of aged skin, and various skin fibroblast activators have been studied. Examples of the fibroblast activators so far reported include a chlorella extract (JP-A-9-40523), hibiscus per se or its extract (JP-A-9-295928), a plant extract of almond, dandelion, bourtree, Cnidium rhizome, Swertia herb, Mulberry bark, Pearch kernel, carrot, hop, Rose of Sharon or Coix laryma-jobi (JP-A-10-36279), water extract of chlorella and aloe vera extract (JP-A-10-36283), a plant extract of sesame, Dioscoreae rhizoma, pepper, Japanese angelica, Tsi or Ophiopogon tuber (JP-A-10-45615), phytoglycogen (JP-A-11-255657) and the like. However, the fibroblast activators so far reported have failed to afford an effective result because they show high effective concentration, poor fibroblast growth rate, high toxicity, problematic safety and the like.

Recently, in addition to the above-mentioned measures for the improvement of wrinkles on the skin, moreover, addition of a substance such as hydroquinone and glycoside thereof, kojic acid and a derivative thereof, ascorbic acid and a derivative thereof, a thiol compound, various animal and plant extracts and the like has been proposed for the purpose of preventing or treating pigmented spot, freckle and the like on the skin. Hydroquinone is used as a pharmaceutical product in Europe and the U.S. In addition, other various skin external preparations for whitening, such as skin external preparations containing alkyl catechol glycoside (JP-A-4-59718), tachioside (JP-A-5-310547), curcumin, capsaicin, 4-hydroxy-3-methoxycinnamaldehyde etc. (JP-A-6-227959), tetraacetyl guaiacol β-D-glucoside (JP-A-6-256138) and the like, are present. However, since these compounds except hydroquinone show effects slowly, the whitening effect is not sufficient. Moreover, hydroquinone and kojic acid have safety problems, and hydroquinone glycoside, kojic acid and a derivative thereof, ascorbic acid and a derivative thereof, and the like have problematically high polarity for use as a cosmetic. Furthermore, thiol compounds such as glutathione, cysteine and the like have problems in the stability after addition. Other presently known animal and plant extracts, for example, placenta extract, aloe extract and the like show insufficient effects.

On the other hand, conagenin is known as a chemotherapeutic agent for cancer (JP-A-2-306953) having very low toxicity for human body. In addition, conagenin has been confirmed to show a platelet and leukocyte increasing action and a systemic side effect alleviation effect (JP-A-5-229939, JP-A-6-65072).

### DISCLOSURE OF THE INVENTION

In view of the above-mentioned situation, the problems to be solved by the present invention is provision of a safe and highly effective fibroblast activator, collagen production promoter, collagen contraction promoter, hyaluronic acid production promoter, ATP production promoter or melanin formation suppressor, and further, a safe and highly effective skin external preparation, cosmetic, pharmaceutical product or food.

Particularly, it is provision of a safe and highly effective skin external preparation, cosmetic, pharmaceutical product or food that can decrease skin wrinkles, improve skin firmness, decrease skin sagging and/or provide a whitening effect.

The present inventors have conducted intensive studies in an attempt to solve the above-mentioned problems and found that conagenin activates human normal fibroblast, promotes collagen production by the human fibroblast, promotes hyaluronic acid production, promotes collagen contraction, and suppresses melanin formation. Based on such findings, they have further studied and found that conagenin is useful as a fibroblast activator, collagen production promoter, collagen contraction promoter, hyaluronic acid production promoter, ATP production promoter or melanin formation suppressor, and that a composition containing the activator, promoter or melanin formation suppressor can be a skin external preparation, cosmetic, pharmaceutical product or food that can improve aging phenomena such as skin wrinkles and sagging, and also a skin external preparation, cosmetic, pharmaceutical product or food having a whitening effect, which resulted in the completion of the present invention.

Accordingly, the present invention provides the following.

(1) A skin external preparation or cosmetic containing at least one kind of compound selected from the group consisting of conagenin, a conagenin derivative, and a pharmaceutically acceptable salt thereof.
(2) A fibroblast activator comprising, as an active ingredient, at least one kind of compound selected from the group consisting of conagenin, a conagenin derivative, and a pharmaceutically acceptable salt thereof.
(3) A collagen production promoter comprising, as an active ingredient, at least one kind of compound selected from the group consisting of conagenin, a conagenin derivative, and a pharmaceutically acceptable salt thereof.
(4) A collagen contraction promoter comprising, as an active ingredient, at least one kind of compound selected from the group consisting of conagenin, a conagenin derivative, and a pharmaceutically acceptable salt thereof.
(5) A hyaluronic acid production promoter comprising, as an active ingredient, at least one kind of compound selected from the group consisting of conagenin, a conagenin derivative, and a pharmaceutically acceptable salt thereof.
(6) An ATP production promoter comprising, as an active ingredient, at least one kind of compound selected from the group consisting of conagenin, a conagenin derivative, and a pharmaceutically acceptable salt thereof.
(7) A melanin formation suppressor comprising, as an active ingredient, at least one kind of compound selected from the group consisting of conagenin, a conagenin derivative, and a pharmaceutically acceptable salt thereof.
(8) A composition for improving wrinkles, which comprises the activator or promoter of any one of the above-mentioned (2) to (6).
(9) A whitening composition comprising the melanin formation suppressor of the above-mentioned (7).
(10) A skin external preparation comprising the activator, promoter or melanin formation suppressor of any one of the above-mentioned (2) to (7).
(11) A cosmetic comprising the activator, promoter or melanin formation suppressor of any one of the above-mentioned (2) to (7).
(12) A pharmaceutical product comprising the activator, promoter or melanin formation suppressor of any one of the above-mentioned (2) to (7).
(13) A food comprising the activator, promoter or melanin formation suppressor of any one of the above-mentioned (2) to (7).
(14) A whitening cosmetic composition comprising, as an active ingredient, at least one kind selected from the group consisting of conagenin, a conagenin derivative and a pharmaceutically acceptable salt thereof.
(15) A whitening agent comprising the composition of the above-mentioned (14).

According to the present invention, a useful fibroblast activator, collagen production promoter, collagen contraction promoter, hyaluronic acid production promoter, ATP production promoter or melanin formation suppressor can be provided. In addition, a skin external preparation, cosmetic (cosmetic composition), pharmaceutical product or food, which is safe and provides a high wrinkle improving effect, can be provided. Moreover, a skin external preparation, cosmetic (cosmetic composition), pharmaceutical product or food, which is safe and provides a high whitening effect, can be provided.

### BRIEF DESCRIPTION OF THE DRAWINGS

FIG. 1 is a graph showing the melanin formation suppressive effects by conagenin and kojic acid in mouse B16 melanoma strain.

### BEST MODE FOR EMBODYING. THE INVENTION

In the present invention, conagenin is a compound represented by the formula (1):

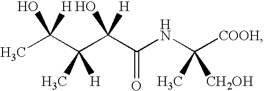

i.e., (2S)—N-[(2R,3S,4R)2,4-dihydroxy-3-methyl-pentanoyl]-2-methylserine. The conagenin used may be naturally-occurring conagenin or chemically synthesized conagenin. The naturally-occurring conagenin can be harvested from a culture of a conagenin-producing microorganism belonging to streptomyces and can be obtained, for example, by the production method described in JP-A-2-306953 and the like.

In addition, the conagenin derivative referred to in the present invention is a compound group represented by the formula (2):

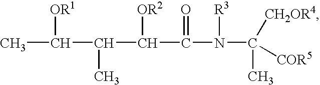

ester forms thereof and ether forms thereof.

In the formula (2), R¹ is hydrogen, a methyl group, an ethyl group or an acyl group represented by the formula: —COR⁶ wherein R⁶ is hydrogen, a methyl group or an ethyl group, R² is hydrogen, a C1-C5 alkyl group, an aralkyl group represented by the formula:

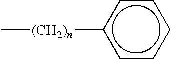

wherein n is an integer of 1-3, or an acyl group represented by the formula: —COR⁷ wherein R⁷ is hydrogen, a methyl group or an ethyl group, R³ is hydrogen, a methyl group or an ethyl group, R⁴ is hydrogen, a C1-C5 alkyl group, an aralkyl group represented by the formula:

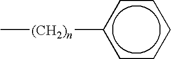

wherein n is an integer of 1-3, or an acyl group represented by the formula: —COR⁸ wherein R⁸ is hydrogen, a methyl group or an ethyl group, R⁵ is an aralkyl group represented by the formula: —OR⁹ wherein R⁹ is hydrogen, a C1-C5 alkyl group or an aralkyl group represented by the formula:

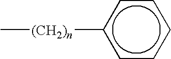

wherein n is an integer of 1-3, or an amino group or substituted amino group represented by the formula: —NHR¹⁰ wherein R¹⁰ is hydrogen, a C1-C5 alkyl group or an aralkyl group represented by the formula:

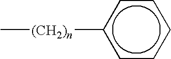

wherein n is an integer of 1-3, provided that R¹, R², R³, R⁴ and R⁵ are not simultaneously hydrogen atoms.

In the compound of the formula (2), the acyl group of the formula —COR⁶, the acyl group of the formula —COR⁷, and the acyl group of the formula —COR⁸ are each preferably a C2-C6 alkanoyl group, more preferably an acetyl group, a propionyl group, a butyryl group or a valeryl group. In addition, preferable examples thereof when R², R⁴, R⁹ and R¹⁰ are aralkyl groups include a benzyl group, a phenethyl group and the like. Preferable examples of the C1-C5 alkyl group include a methyl group, an ethyl group, an n-propyl group, an iso-propyl group, an n-butyl group, an iso-butyl group, a t-butyl group, a pentyl group and the like. These conagenin derivatives can be obtained by the production method described in JP-A-4-187664 and the like.

Examples of the ester form of the above-mentioned conagenin derivative include ester forms of phosphoric acid, sulfuric acid, fatty acid and the like, which can be obtained by esterification of the above-mentioned conagenin derivative by a known method. Examples of the ether form include an ether form of sugar, which can be obtained by a known sugar introduction method.

The aforementioned conagenin or a conagenin derivative may form a salt. Of the salts formed, a pharmaceutically acceptable salt can be used. While the aforementioned pharmaceutically acceptable salt is not particularly limited, for example, a salt of conagenin, a salt of a conagenin derivative at the carboxyl group and the like can be used. Examples thereof include alkali metal salts such as sodium salt, potassium salt, lithium salt and the like, alkaline earth metal salts such as calcium salt, magnesium salt and the like, metal salts such as aluminum salt, iron salt, zinc salt, copper salt, nickel salt, cobalt salt and the like, inorganic salts such as ammonium salt and the like, organic salts such as amine salts (e.g., t-octylamine salt, dibenzylamine salt, morpholine salt, glucosamine salt, phenylglycine alkyl ester salt, ethylenedimine salt, N-methylglucamine salt, guanidine salt, diethylamine salt, triethylamine salt, dicyclohexylamine salt, N,N′-dibenzylethylenediamine, chloroprocaine salt, procaine salt, diethanolamine salt, N-benzyl-phenethylamine salt, piperazine salt, tetramethylammonium salt, tris(hydroxymethyl)aminomethane salt and the like), and the like, hydrohalic acid salts such as hydrogen fluoride, hydrochloride, hydrogen bromide, hydroiodide and the like, inorganic acid salts such as nitrate salt, perchlorate, sulfate, phosphate and the like, lower alkanesulfonate such as methanesulfonate, trifluoromethanesulfonate, ethanesulfonate and the like, arylsulfonate such as benzenesulfonate and the like, organic acid salts such as acetate, malate, fumarate, succinate, citrate, tartrate, oxalate, maleate and the like, amino acid salts such as glycine salt, lysin salt, arginine salt, ornithine salt, glutamic acid salt, aspartic acid salt and the like, and the like. In addition, pharmaceutically acceptable salts of conagenin and conagenin derivative may become hydrates. Such salts are also encompassed in the pharmaceutically acceptable salt.

Conagenin shows a strong human fibroblast activating action, as shown in the below-mentioned evaluation test, has a collagen production promoting action, a collagen contraction promoting action, a hyaluronic acid production promoting action, and an ATP production promoting action, and also a melanin formation suppressive action (strongly suppresses melanin formation in a blackening suppress test using mouse B16 melanoma). In addition, it characteristically shows very low toxicity. Accordingly, conagenin, a conagenin derivative and a pharmaceutically acceptable salt thereof (hereinafter collectively referred to as “conagenin compound”) are useful as fibroblast activators, collagen production promoters, collagen contraction promoters, hyaluronic acid production promoters, ATP production promoters or melanin formation suppressors, and an activator, a promoter and a melanin formation suppressor comprising such conagenin compound can be utilized as a skin external preparation, cosmetic, pharmaceutical product or food for the purpose of the prophylaxis or improvement of skin wrinkles, sagging and the like, or producing firm skin and the like (i.e., as composition for improving wrinkles), or a skin external preparation, cosmetic, pharmaceutical product or food for the prophylaxis or improvement of pigmented spot, freckle and the like (i.e., as whitening composition).

It is considered that melanin is formed through the pathway of tyrosine→dopa→dopaquinone→dopachrome→5,6-dihydroxyindole→melanin, and its formation can be suppressed by inhibiting the activity of the enzymes that act through an oxidation step of tyrosine→dopa, dopa→dopaquinone, such as tyrosinase, Trp-1, Trp-2 and the like (Osamu Okuda, Shuji Saito, Kazunari Suzuki, “Koryo to Keshohin no Kagaku” p 266, 1982, Hirokawa Shoten, Tokyo).

Furthermore, since conagenin compound not only promotes component secretion and the like of skin compositions such as collagen, hyaluronic acid, elastin and the like, but also activates cell metabolism of cells such as fibroblast, epidermal cell, basal epidermal cell and the like, the effect of promotion of cell turnover, cell growth and the like is also expected of the skin external preparation, pharmaceutical product, cosmetic and food of the present invention.

In the present invention, the conagenin compound may be a fraction obtained by extracting an active ingredient, which is harvested from a culture of a chemically synthesized substance or a producing microorganism, with an organic solvent. However, a purified preparation obtained by purifying the oily substance by silica gel column, high performance liquid chromatography and the like is preferable.

When the skin external preparation or cosmetic of the present invention is a composition for improving wrinkles, the content of the conagenin compound (i.e., the fibroblast activator, collagen production promoter, collagen contraction promoter, hyaluronic acid production promoter or ATP production promoter of the present invention) is generally 0.00000001-50%, preferably 0.0001-10%, of the total composition weight. When it is a whitening composition, the content of the conagenin compound (i.e., the melanin formation suppressor of the present invention) is generally 0.00001-20%, preferably 0.001-20%, of the total composition weight.

The skin external preparation or cosmetic of the present invention can appropriately contain, besides the conagenin compound (i.e., the fibroblast activator, collagen production promoter, collagen contraction promoter, hyaluronic acid production promoter, ATP production promoter or melanin formation suppressor of the present invention), various components generally used for the below-mentioned pharmaceutical product, cosmetic and the like. In addition, when a skin external preparation or cosmetic is particularly a composition for improving wrinkles, a known anti-aging component or anti-wrinkle agent can also be contained. While the anti-aging component or anti-wrinkle agent in this case is not particularly limited, it is, for example, vitamin C, vitamin C derivative, ceramide, α-hydroxy acid, retinol acid, estrogen-like substance, mucoperiosteum fragmentation suppressor, active oxygen scavenger, antioxidant, hyaluronic acid, hyaluronan-degrading enzyme inhibitor, collagen, collagen resolvent, collagenolytic inhibitor, elastin, elastin production promoter, elastase inhibitor, nucleic acid, whitening agent and the like, as well as known skin fibroblast activator, hyaluronic acid production promoter, collagen production promoter and the like other than the conagenin compound. In addition, when a skin external preparation or cosmetic is particularly a whitening composition, it can also be used as a mixture with other components having a whitening effect. Examples of other components having a whitening effect include vitamin C derivatives such as ascorbic acid, ascorbic acid glucoside and the like, arbutin, tachioxide, 3,4-dim ethoxyphenyl-O-D-glucose, kojic acid, hydroquinone, L-cysteine, mulberry extract, licorice extract and the like.

The skin external preparation or cosmetic of the present invention is a pharmaceutical product • cosmetic-related product, and can take various dosage forms. That is, it includes cosmetics such as lotion, emulsion, cream, packing agent, powder, foundation, sun care, toner, ointment, aerosol, emulsion, gel, soap and the like, toiletry products such as shampoo, rinse, soap, body shampoo and the like, and skin external preparations as pharmaceutical products such as lotion, essence, emulsion, cream, ointment and the like. Moreover, it can be used as an adhesive preparation and a bath agent. When the efficacy of a quasi drug includes a category relating to wrinkle, sagging or firmness or a category relating to whitening, it also includes medical cosmetics. That is, “the skin external preparation or cosmetic of the present invention” includes pharmaceutical products, cosmetics, toiletry products and quasi drugs for external use for the skin.

When an ointment is to be produced, a carrier generally used for ointments such as base, stabilizer, wetting agent, preservative and the like is added as necessary, mixed by a conventional method, and made into a preparation. Examples of the aforementioned base include liquid paraffin, white petrolatum, white beeswax, octyldodecyl alcohol, paraffin and the like. Examples of the preservative include methyl parahydroxybenzoate, ethyl parahydroxybenzoate, propyl parahydroxybenzoate and the like.

When an adhesive preparation is to be produced, it is produced by coating a support generally used for adhesive preparations with the aforementioned ointment, paste preparation, cream preparation, gel preparation and the like by a conventional method. Examples of a desirable support include a film and a foam sheet made of cotton, staple fiber, woven fiber made of a chemical fiber, non-woven fabric, soft vinyl chloride, polyethylene, polyurethane and the like.

The skin external preparation and cosmetic of the present invention can be produced by using, where necessary, components and additives used for pharmaceutical product, quasi drug, cosmetic and the like in combination, to the extent that the effect of the invention is not impaired. Specific examples of the addition components include the following.

Examples of the surfactant include anion surfactants such as soap base, fatty acid soap, higher alkylsulfate, alkyl ether sulfate, N-acylsarcosinate, higher fatty acid amide sulfonate, phosphate, sulfosuccinate, alkylbenzene sulfonate, N-acylglutamate, higher fatty acid ester sulfate, sulfonated oil, POE (polyoxyethylene) alkyl ether carboxylate, POE alkylallyl ether carboxylate, α-olefin sulfonate, higher fatty acid ester sulfonate, secondary alcohol sulfate, higher fatty acid alkylol amide sulfate, lauroyl monoethanol amide succinate, N-palmitoyl aspartate ditriethanolamine, casein sodium and the like, cation surfactants such as alkyltrimethylammonium salt, dialkyldimethylammonium salt, alkylpyridium salt, alkylquaternaryammonium salt, alkyldimethylbenzylammonium salt, alkylisoquinolinium salt, dialkylmorpholium salt, POE alkylamine, alkylamine, polyamine fatty acid derivative, amylalcohol fatty acid derivative, benzalkonium chloride, benzethonium chloride and the like, ampholytic surfactant such as imidazoline surfactant, betaine surfactant and the like, lipophilic nonionic surfactants such as sorbitan fatty acid ester, glycerin fatty acid ester, propylene glycol fatty acid ester, hydrogenated castor oil derivative, glycerin alkyl ether, polyoxyethylene• methylpolysiloxane copolymer and the like, lipophilic nonionic surfactants such as POE sorbitan fatty acid ester, POE sorbit fatty acid ester, POE glycerin fatty acid ester, POE fatty acid ester, POE alkyl ether, POE alkyl phenyl ether, POE-POP alkyl ether, tetraPOE•tetraPOP ethylenediamine condensate, POE hydrogenated castor oil derivative, POE beeswax-lanolin derivative, alkanolamide, POE propyleneglycol fatty acid ester, POE alkylamine, POE fatty acid amide, sucrose fatty acid ester and the like, and the like.

Examples of the oils include animal vegetable oil and hardened oil thereof such as avocado oil, olive oil, sesame oil, camellia oil, evening primrose oil, turtle oil, macadamia nut oil, corn oil, mink oil, rapeseed oil, egg-yolk oil, apricot kernel oil, wheat germ oil, sasanqua oil, castor oil, linseed oil, safflower oil, cottonseed oil, perilla oil, soybean oil, peanut oil, carmelia sinesis oil, Japanese torreya seed oil, rice bran oil, tung oil, jojoba oil, cacao butter, palm oil, horse oil, palm oil, elaesis guineensis kernel oil, beef tallow, mutton tallow, lard, lanolin, whale wax, beeswax, carnauba wax, Japan wax, candelilla wax, squalane and the like, mineral oils such as liquid paraffin, vaseline and the like, synthetic triglycerols such as tripalmitate glycerin and the like, other oily components and the like.

Examples of the higher fatty acid include lauric acid, myristic acid, palmitic acid, oleic acid, linoleic acid, linolenic acid, stearic acid, behenic acid, 12-hydroxystearic acid, isostearic acid, undecyne acid, tall oil acid, eicosapentaenoic acid, docosahexaenoic acid and the like. Examples of the higher alcohol include lauryl alcohol, cetyl alcohol, stearyl alcohol, bechenyl alcohol, myristyl alcohol, oleyl alcohol, cetostearyl alcohol, jojoba alcohol, lanolin alcohol, batyl alcohol, 2-decyltetratecesinol, cholesterol, phytosterol, isostearyl alcohol and the like. Examples of the synthetic esters include cetyl octanoate, octyldodecyl myristate, isopropyl myristate, myristyl myristate, isopropyl palmitate, butyl stearate, hexyl laurate, decyl oleate, dimethyl octanoate, cetyl lactate, myristyl lactate and the like. Examples of the silicone include chain polysiloxane such as dimethyl polysiloxane, methylphenyl polysiloxane and the like, cyclic polysiloxane such as decamethyl cyclopolysiloxane and the like, one having a three dimensional net structure such as silicone resin and the like, and the like.

Examples of the moisturizing agent include glycerin, propylene glycol, 1,3-butylene glycol, dipropylene glycol, polyethylene glycol, hexylene glycol, xylitol, sorbitol, maltitol, chondroitin sulfuric acid, hyaluronic acid, mucoitinsulfuric acid, atelocollagen, urea, sodium lactate, bile salt, dl pyrrolidone carboxylate, soluble collagen, atelocollagen, collagen resolvent, hyaluronic acid, hyaluronic acid resolvent, nucleic acid and the like, as well as various animal and plant extracts, yeast extract and the like.

Examples of the UV absorber include benzoic acid UV absorbers such as paraamino benzoic acid, paraamino benzoic acid derivative and the like, antranilic acid UV absorbers such as homomentyl N-acetylanthranilate and the like, salicylic acid UV absorbers such as amyl salicylate and the like, cinnamic acid UV absorbers such as octyl cinnamate and the like, benzophenone UV absorbers such as 2,4-dihydroxybenzophenone and the like, 4-methylbenzylidenecamphor, 3-benzylidenecamphor, 2-phenyl-5-methylbenzoxazole, nucleic acid and the like.

Examples of the vitamins include vitamin A such as vitamin oil, retinol and the like, vitamin B2 such as riboflavin and the like, vitamin B6 such as prydoxine hydrochloride and the like, vitamin C such as L-ascorbic acid and the like, pantothenic acids such as calcium pantothenate and the like, vitamin D such as ergocalciferol and the like, nicotinic acids such as nicotinic acid amide and the like, vitamin E such as tocophenol acetate and the like, vitamin P, biotin and the like.

Examples of the natural aqueous polymer include gum arabic, gum tragacanth, galactan, gua gum, carob gum, karaya gum, carageenan, pectin, agar, Pyrus cydonia seed, argecolloid, starch, xanthan gum, dextran, succinoglucan, pullulan, collagen, casein, hyaluronic acid, albumin, gelatin and the like. Examples of the semi-synthetic aqueous polymer include cellulose polymers such as methylcellulose, nitrocellulose, carboxymethylcellulose sodium and the like, starch polymers such as carboxymethyl starch and the like, polymer alginate such as sodium alginate and the like, and the like. Examples of the synthetic aqueous polymer include vinyl polymers such as polyvinyl alcohol, carboxyvinyl polymer and the like, polyoxyethylene polymers such as polyethylene glycol 2000 and the like, copolymerized polymers such as polyoxyethylene polyoxypropylene copolymer and the like, acrylic polymers such as polyacrylamide and the like, polyethylenimine, cation polymer and the like.

Examples of the powder component include inorganic powders such as talc, kaolin, mica, sericite, magnesium carbonate, calcium carbonate, silicate, silica, barium sulfate, exsiccated gypsum, fluorapatite, ceramic powder and the like, organic powders such as nylon powder, polyethylene powder, polystyrene powder, cellulose powder and the like, and the like. Examples of the dye agent include inorganic pigments such as titanium dioxide, iron oxide, carbon black, cobalt violet and the like, organic pigments such as Red 201, Red 3, Yellow 205, Yellow 4 and the like, natural dyes such as chlorophyll, riboflavin, β-carotene, astaxanthin, lycopene and the like, plant extract dye such as safflower, turmeric and the like, and the like. Examples of the preservative include benzoate, salicylate, sorbate, dehydroacetate, paraoxybenzoate, benzalkonium chloride, hinoki thiol, resorcin, ethanol and the like. Examples of the antioxidant include tocophenol, ascorbic acid, butylhydroxyanisole, dibutylhydroxytoluene, gallic acid ester and the like. Examples of the chelating agent include sodium ethylenediamine tetraacetate, sodium polyphosphorate, citric acid and the like.

Moreover, a plant extract having a bioactive action such as antibacteria, cell activation, sebum secretion adjustment, anti-inflammation, astringency, antioxidization, whitening, active oxygen suppress, antiallergy and the like and an extract fraction or purified product thereof can also be used in combination. Besides those mentioned above, flavor, alcohols such as lower alcohol, polyvalent alcohol and the like, hydrocarbon, silicone, thickener, film agent, metal ion sealant, saccharides, amino acids, organic amines, synthesized resin emulsion, pH adjusting agent, skin nutritional supplement, antioxidant aids, preservative, fungicide, buffer, water and the like can be appropriately combined.

In the present invention, the administration pathway of the conagenin compound (i.e., fibroblast activator, collagen production promoter, collagen contraction promoter, hyaluronic acid production promoter, ATP production promoter or melanin formation suppressor of the present invention) is mainly a parenteral one represented by the aforementioned skin external preparation or cosmetic as being effective for the improvement of skin wrinkles, sagging, firmness and the like or whitening thereof. However, oral ingestion (administration) by making a preparation such as liquid (drink agent), paste agent, powder agent, granule, capsule, tablet, syrup, inhalant and the like is also possible. In addition, intravenous administration of injection is also possible. In other words, the present invention provides a pharmaceutical product such as an agent for oral administration, intravenous administration and the like (i.e., pharmaceutical product other than skin external pharmaceutical product), which contains the conagenin compound (i.e., fibroblast activator, collagen production promoter, collagen contraction promoter, hyaluronic acid production promoter, ATP production promoter or melanin formation suppressor of the present invention). In the pharmaceutical product of the present invention such as an agent for oral administration, intravenous administration and the like, the content of the conagenin compound is not particularly limited. For example, when an agent for oral administration is a composition for the improvement of wrinkles, the content is generally 0.0000001-50%, preferably, 0.0001-10%, of the total composition weight, and when the agent is a whitening composition, the content is generally 0.00001-20%, preferably 0.001-10%, of the total composition weight.

The pharmaceutical product of the present invention (i.e., pharmaceutical product other than skin external pharmaceutical product) can contain various addition components such as excipient, stabilizer, wetting agent, emulsifier, absorption promoter, pH adjusting agent, surfactant, diluent, carrier and the like. Specific examples of these addition components particularly include starch, saccharides such as lactose, magnesium sulfate, talc, gelatin, cellulose derivative such as hydroxypropylcellulose, vegetable oil such as soybean oil and sesame oil, animal oil, synthetic oil, rubber, water such as saline and the like, alcohols such as ethanol, 1,3-butylene glycol, polyalkylene glycol and the like, and the like. In addition, a component selected from those that can be added to the aforementioned skin external preparation and cosmetic may be added.

In the present invention, the conagenin compound (i.e., fibroblast activator, collagen production promoter, collagen contraction promoter, hyaluronic acid production promoter, ATP production promoter or melanin formation suppressor of the present invention) can be added to ordinary foods and drinks such as confectionery, bakery, cereal preparations, dairy products, oil and fat products, soft drinks, powder drinks, seasonings and the like, and articles of taste (including what is called “health food (including those by the names of dietary supplement, health supplement, supplement and the like)”). That is, the present invention also provides a food containing the conagenin compound (i.e., fibroblast activator, collagen production promoter, collagen contraction promoter, hyaluronic acid production promoter, ATP production promoter or melanin formation suppressor of the present invention).

Moreover, the food of the present invention can contain various addition components such as sweetener, acidulant, preservative, flavor, colorant, excipient, stabilizer, wetting agent, emulsifier, absorption promoter, pH adjusting agent, surfactant, diluent, carrier and the like. In addition, a component selected from those that can be added to the aforementioned skin external preparation and cosmetic may be added.

The food of the present invention can be prepared by a method similar to that for general food except addition of the conagenin compound (i.e., fibroblast activator, collagen production promoter, collagen contraction promoter, hyaluronic acid production promoter, ATP production promoter or melanin formation suppressor of the present invention).

### EXAMPLES

The present invention is explained in more detail in the following by referring to Examples, which are not to be construed as limitative.

Conagenin used in the following Examples was prepared based on the production method described in JP-A-2-306953. Specifically, Streptomyces roseosporus MI696-AF3 (FERM BP-2738) was cultured, activated carbon was added to the filtrate of the obtained culture medium, the active ingredient adsorbed onto the activated carbon was extracted with an organic solvent, the oily substance obtained by concentration was purified by silica gel column, thin layer chromatography or high performance liquid chromatography to give conagenin.

### Example 1

The present inventors performed a fibroblast activating action evaluation test by the MTT reduction method and using normal skin fibroblasts derived from human (see TIM Mosmann; Journal of Immunological Methods p 55-63, 1983).

### <Test Method>

Using DMEM (Gibco) containing 5% FBS (fetal bovine serum; purchased from NICHIREI CORPORATION), normal skin fibroblasts derived from human (manufactured by KURABO INDUSTRIES LTD.) were plated on a 96 well plate at a density of 2×10⁴ cells/well, and cultured at 37° C., 5% CO₂ for 24 hr. After removing the medium, the cells were washed with PBS(−) (NISSUI PHARMACEUTICAL CO., LTD.), the medium was changed to DMEM containing 1% FBS supplemented with each concentration of conagenin, and the cells were cultured at 37° C., 5% CO₂. The blank was MEM containing 1% FBS, which was free of test specimen. After culture for 48 hr, the absorbance at 550 nm was measured by MTT reduction method, based on which the MTT reduction amount was determined. The results are shown in Table 1. The cell activation rate was shown in a percentage relative to the absorbance of additive-free cultured cell (control) as 100.

TABLE 1

| | addition | | |
|---|---|---|---|
| | concentration | | cell activation rate |
| control | — | | 100% |
| conagenin | 0.01 | μM | 105% |
| | 0.05 | μM | 120% |
| | 0.1 | μM | 125% |
| | 1 | μM | 111% |
| | 10 | μM | 113% |
| | 40 | μM | 124% |
| | 80 | μM | 113% |
| | 160 | μM | 113% |
| | 310 | μM | 113% |

As is clear from Table 1, addition of the test substance resulted in higher cell activation rates as compared to the absence of the test substance. Therefore, the test substance is considered to have activated normal fibroblasts derived from human skin.

### Example 2

### Type I Collagen Production Promoting Effect of Conagenin on Human Skin Fibroblasts

Normal human skin fibroblasts (manufactured by KURABO INDUSTRIES LTD.) were added to a 24 well plate at 5×10⁴ cells per well. Using Medium 106S medium (manufactured by KURABO INDUSTRIES LTD.) containing 2% FBS, the cells were cultured under an atmosphere of 95% (V/V) air-5% (V/V) carbon dioxide gas at 37° C. for 24 hr. The medium was changed to Medium 106S medium (free of FBS) containing each sample, and the cells were cultured under the same conditions for 24 hr. After the completion of culture, the culture supernatant was taken to evaluate the type I collagen biosynthesizability and, to count the cells, the cells were harvested by a trypsin treatment. The samples used were two kinds of conagenin contents of 0.1 μM, 1 μm, 10 μM, 100 μM, 1 mM (final concentration), and magnesium L-ascorbyl phosphate (manufactured by Wako Pure Chemical Industries, Ltd., final concentration 100 μM) as a positive control (one added with PBS instead of the sample was used as a negative control). The production of type I collagen by the cells was evaluated by measuring the amount of type I procollagen C terminal peptide (P (to be abbreviated as Procollagen Type I C-peptide: abbreviated as PIP) secreted in the culture supernatant. Specifically, the measurement was performed using a PIP measurement kit (manufactured by TAKARA BIO INC.) and according to the protocol attached thereto. The amount of the collagen produced (shown in a relative value to collagen production amount of negative control as 100), and the number of cells per well are shown in Table 2. The cells were visually counted after detaching them with trypsin from the plate after the test (each sample addition group subjected to the test was n=3, and the results show their average values).

TABLE 2

| | produced | |
|---|---|---|
| | amount of | number of |
| | collagen | cells |
| negative control | 100 | 4.9 × 10⁵ |
| conagenin 0.1 μM | 196 | 5.1 × 10⁵ |
| conagenin 1 μM | 197 | 5.1 × 10⁵ |
| conagenin 10 μM | 204 | 5.2 × 10⁵ |
| conagenin 100 μM | 208 | 4.8 × 10⁵ |
| conagenin 1 mM | 202 | 4.7 × 10⁵ |
| positive control | 152 | 4.6 × 10⁵ |

From Table 2, it has been clarified that conagenin has a superior collagen production promoting effect. From these results, it has been clarified that conagenin has a superior collagen contraction promoting action and can exhibit a superior effect on skin wrinkles and sagging.

### Example 3

### Collagen Contraction Promoting Effect of Conagenin for Human Skin Fibroblasts

A collagen solution (collagen was used trade name I-AC manufactured by KOUKEN Co., Ltd.) dissolved in normal human skin fibroblasts (manufactured by KURABO INDUSTRIES LTD., 1×10⁵ cells/ml) was prepared on ice according to the explanation insert attached to the product, and the collagen was gelled in 6 wells at 37° C. Thereafter, 0.25% FBS/DMEM medium containing a sample (PBS as conagenin or negative control) was added. The gel was detached from the dish wall, and the level of contraction of the gelled collagen was examined. As a positive control, DMEM medium containing 10% FBS instead of 0.25% FBS was used. The medium was changed every 2 days and, one week later, the medium was removed by suction. The diameter of the collagen gel was measured, the area was calculated, and the area ratio was compared to that of the negative control as 1. The results are shown in Table 3.

TABLE 3

| | collagen gel area ratio |
|---|---|
| negative control | 1 |
| positive control | 0.47 |
| conagenin 1 μM | 0.47 |
| conagenin 100 μM | 0.44 |
| conagenin 5 mM | 0.42 |

From these results, it has been clarified that conagenin, the active ingredient of the skin external preparation of the present invention, has a superior collagen contraction promoting action and can exhibit a superior effect on skin wrinkles and sagging.

### Example 4

### Evaluation of Action on Type I Collagen Gel Contractability after Glucose Modification in Human Skin Fibroblasts

A collagen solution (collagen was used trade name I-AC, manufactured by KOUKEN Co., Ltd.) was prepared on ice according to the explanation insert attached to the product, and the collagen was gelled in 12 wells at 37° C. A glucose-6-phosphate solution was added to the final concentration of 100 mM, and the mixture was incubated at 37° C. for 7 days to allow glycation reaction. Unreacted glucose-6-phosphate was removed, 1×10⁵ cells/ml of fibroblast was sown on the collagen gel, and cultured for 5 hr using a 0.25% FBS/DMEM medium. The medium was removed, and collagen or 0.25% FBS/DMEM medium containing PBS as negative control was added. The gel was detached from the dish wall, and the level of contraction of the gelled collagen was examined. As a positive control, DMEM medium containing 10% FBS was used. The medium was changed every 2 days and, one week later, the medium was removed by suction. The diameter of the collagen gel was measured, the area was calculated, and the area ratio was compared to that of the negative control as 1. The results are shown in Table 4.

TABLE 4

| | collagen gel area ratio |
|---|---|
| negative control | 1 |
| positive control | 0.36 |
| conagenin 1 μM | 0.81 |
| conagenin 100 μM | 0.64 |
| conagenin 5 mM | 0.49 |

From these results, it has been clarified that conagenin, the active ingredient of the skin external preparation of the present invention, has a superior glycated collagen contraction promoting action and can exhibit a superior effect on skin wrinkles and sagging.

As mentioned above, conagenin is found to have an action to quantitatively (collagen production promoting activity) or qualitatively (collagen contraction promoting action) maintain and enhance collagen that constitutes the main fiber structure in the dermis. It has been clarified that using conagenin as the active ingredient of a skin external preparation, the aging phenomenon in the skin structure (typically, wrinkles and sagging) can be effectively prevented or improved.

### Example 5

### Hyaluronic Acid Production Promoting Effect of Conagenin for Human Fibroblasts

The number of cells of normal human fibroblasts was adjusted to 5.0×10⁴ cells/mL in Medium 106S medium containing 2% FBS, and plated on a 24 well plate by 1.0 mL. The cell was cultured under an atmosphere of 95% (V/V) air-5% (V/V) carbon dioxide gas at 37° C. for 24 hr, and then Medium 106S medium (free of FBS) containing various concentrations of conagenin was added. After further culture for 72 hr, the culture supernatant was collected, and the concentration of hyaluronic acid released into the medium was measured by an inhibitory method (hyaluronic acid measurement kit, manufactured by SEIKAGAKU CORPORATION) utilizing a hyaluronic acid-binding protein. The method of operation was as indicated in the attached explanation insert. The results obtained using PBS as a negative control instead of conagenin was taken as 100 and the amount ratio of hyaluronic acid in the medium was calculated. The results are shown in Table 5 (each sample addition group subjected to the test was n=3, and the results show their average values).

TABLE 5

| | amount of | |
|---|---|---|
| | hyaluronic acid | |
| | produced | number of cells |
| negative control | 100 | 5.4 × 10⁵ |
| conagenin 1 μM | 108 | 5.3 × 10⁵ |
| conagenin 10 μM | 120 | 5.5 × 10⁵ |
| conagenin 100 μM | 132 | 5.3 × 10⁵ |
| conagenin 1 mM | 147 | 5.1 × 10⁵ |
| conagenin 5 mM | 199 | 4.0 × 10⁵ |
| positive control | 131 | 4.7 × 10⁵ |

From these results, it has been clarified that conagenin increases the amount of hyaluronic acid.

### Example 6

### ATP Production Promotion

The evaluation was performed according to the following procedure. Normal human skin fibroblasts (manufactured by KURABO INDUSTRIES LTD.) were plated on a 96 well microplate at 2.0×10⁴ cells per well. As the medium for plating, commercially available Medium 106S (manufactured by KURABO INDUSTRIES LTD.) containing 2% FBS, heparin 10 μg/ml and hydrocortisone 1 μg/ml was used. After culture for 24 hr, the medium was changed to a medium supplemented with various concentrations of conagenin, and the cells were cultured for 48 hr.

Then, the medium was removed from the 96 well microplate, and the amount of ATP synthesized in the cell was measured using a ATP measurement kit (ATP Lite, manufactured by PerkinElmer, Inc.). That is, the cells were washed with PBS(−), the cell membrane was lysed with a Lysis solution, a luminescence substrate was added, and the mixture was transferred to a black 96 well microplate (View Plate). The chemical luminescence was measured with a luminometer. The effect of the sample was evaluated using, as an index, the average ATP amount without addition of the sample as 100. For statistical processing, parametric multiple comparison (Dunnett Type) relative to control group without addition of the sample was performed, wherein a risk rate of less than 5% is indicated with *, a risk rate of less than 1% is indicated with ** and a risk rate of less than 0.1% is indicated with ***.

TABLE 6
intracellular ATP amount index (n = 4, mean ± standard deviation)

| | | ATP amount |
|---|---|---|
| | | (average ATP amount |
| conagenin | | without addition of |
| concentration | | sample is 100) |
| 0 | μM | 100 ± 6 |
| 0.20 | μM | 120 ± 18 |
| 0.39 | μM | 146 ± 30*** |
| 0.78 | μM | 128 ± 8* |
| 1.56 | μM | 133 ± 8** |
| 3.13 | μM | 150 ± 20*** |
| 6.25 | μM | 155 ± 26*** |
| 12.5 | μM | 160 ± 7*** |
| 25 | μM | 153 ± 4*** |
| 50 | μM | 168 ± 13*** |
| 100 | μM | 164 ± 16*** |

As is clear from the Table, a clear ATP production promoting action of conagenin on skin fibroblasts was found. Particularly, when conagenin was added at not less than 0.39 μM, a statistically significant ATP production promoting action was observed as compared to that without the addition.

### Example 7

### Melanin Formation Suppression Test

A melanin formation suppression test was performed as follows using mouse melanoma cells. First, 2×10⁴ B16 melanoma cells were plated in a 35 mm diameter dish containing Eagle's minimum nutrition medium (3 ml) supplemented with 10% (v/v) fetal bovine serum, and cultured in a carbon dioxide incubator adjusted to 5% (v/v) carbon dioxide gas at 37° C. for about 24 hr. Then, pure water or a sample dissolved in pure water was added thereto to a final concentration of 0.1 mM, 1.0 mM or 10 mM. As a positive control, kojic acid (manufactured by Sigma Ltd.) was added to the same concentration. The cells were cultured for 5 more days under the same conditions, treated with trypsin, and centrifuged to collect the cells in a 1.5 ml Eppendorf. The level of whitening of the cells was visually evaluated, and indicated: whitening high→++; whitening moderate→+; somewhat whitened→+−; no whitening→−. Simultaneously, changes in the cell mass volume was visually evaluated and used as a cytotoxicity index.

Suppression of blackening by conagenin was measured by this measurement method. As shown in Table 7, conagenin was found to highly suppress blackening, with no change in the cell mass volume, thus demonstrating high whitening effect with low toxicity. On the other hand, while kojic acid showed effect to a certain degree, cell death occurred by the addition at 10 mM.

TABLE 7

| | sample | | |
|---|---|---|---|
| | concentration | level of | change in cell |
| | (mM) | whitening | mass volume |
| water | | − | none |
| conagenin | 10 | ++ | none |
| | 1 | ++ | none |
| | 0.1 | + | none |
| kojic acid | 10 | immeasurable | yes (cell death) |
| | 1 | + | none |
| | 0.1 | +− | none |

### Example 8

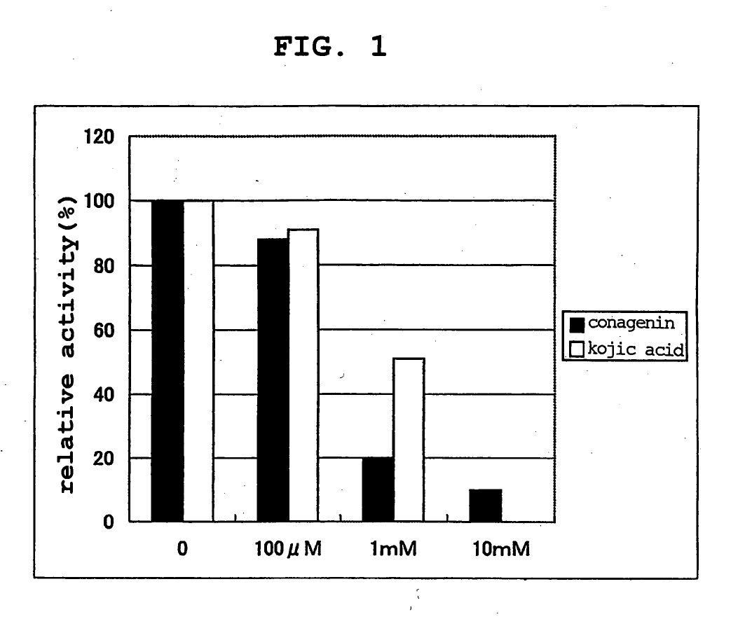

To the cells recovered in Example 7 was added 100 μl of cell suspension (PBS buffer containing Triton X-100 at 0.1% (v/v)). After thorough suspending, the suspension was stood at 4° C. for 1 hr. Thereto was added 100 μl of 10 mM L-DOPA (manufactured by Nacalai Tesque), and the mixture was incubated at 37° C. for 1 hr. Thereafter, the absorbance at 495 nm was measured by a plate reader (multiscanplus MKII, manufactured by Japan Flow Laboratory), and the melanin formation amount was measured. The results are shown in FIG. 1.

As shown in FIG. 1, it has been clarified that conagenin suppresses melanin formation at a low concentration (kojic acid could not be measured since, as is clear from Example 7, cell death occurred at 10 mM).

### Example 9

### Melanin Formation Suppression Test

A melanin formation suppression test was performed using human melanocytes (manufactured by KURABO INDUSTRIES LTD.). That is, human normal melanocytes amplified in Medium 254 medium (human melanocyte medium added with HMGS, manufactured by KURABO INDUSTRIES LTD.) was added to a 96 well plate at 10⁴ cells per well, and cultured in a carbon dioxide incubator adjusted to 5% (v/v) carbon dioxide gas at 37° C. for about 24 hr. Then, pure water or a sample (conagenin) dissolved in pure water was added thereto to a final concentration of 0.1 mM, 1.0 mM or 10 mM. As a positive control, kojic acid (manufactured by Sigma Ltd.) was added to the same concentration. The cells were cultured for 5 more days under the same conditions. To the recovered cells was added 50 μl of cell suspension (PBS buffer containing Triton X-100 at 0.1% (v/v)). After thorough suspending, the suspension was stood at 4° C. for 1 hr. Thereto was added 50 μl of 10 mM L-DOPA (manufactured by Nacalai Tesque), and the mixture was incubated at 37° C. for 1 hr. Thereafter, the absorbance at 495 nm was measured by a plate reader (multiscanplus MKII, manufactured by Japan Flow Laboratory), and the melanin formation amount was measured (shown in a relative value to the value without addition of sample as 100).

As shown in Table 8, it has been clarified that conagenin suppresses melanin formation at a low concentration (kojic acid could not be measured since, as is clear from Example 7, cell death occurred at 10 mM).

TABLE 8

| | 100 μM | 1 mM | 10 mM |
|---|---|---|---|
| conagenin | 92 | 66 | 17 |
| kojic acid | 105 | 88 | — |

### Example 10

The formulation of skin lotion is shown in Table 9. The components (1)-(7) in Table 9 were mixed with (9), homogeneously dissolved, (8) was added and mixed therewith and (9) was added to make the total amount 100 wt %.

TABLE 9

| component | | amount added (wt %) |
|---|---|---|
| (1) | polyoxyethylene(20)sorbitan | 1 |
| | monolaurate | |
| (2) | 1,3-butylene glycol | 3 |
| (3) | sorbitol | 2 |
| (4) | sodium pyrrolidonecarboxylate | 3 |
| (5) | conagenin | 1 |
| (6) | ethanol | 10 |
| (7) | methyl parahydroxybenzoate | 0.1 |
| (8) | flavor | 0.2 |
| (9) | purified water | q.s. |

| | |
|---|---|
| total amount | 100 |

### Example 11

The formulation of skin emulsion is shown in Table 10. The oil phase components (1)-(5) in Table 10 were mixed, homogeneously dissolved and heated to 75° C. On the other hand, aqueous phase components (6), (7), (9), (10), (13) were mixed, dissolved and heated to 75° C. Then, the oil phase components were added to the above-mentioned aqueous phase components and preliminarily emulsified. (8) was added and uniformly emulsified in a homomixer. The mixture was cooled, (10) was added to adjust pH, (12) was added at 50° C. and mixed.

TABLE 10

| component | | amount added (wt %) |
|---|---|---|
| (1) | squalane | 5 |
| (2) | white petrolatum | 2 |
| (3) | beeswax | 0.5 |
| (4) | sorbitan sesquioleate | 0.8 |
| (5) | polyoxyethylene(20)oleyl ether | 1.2 |
| (6) | propylene glycol | 5 |
| (7) | ethanol | 5 |
| (8) | 1.0 wt % aqueous carboxyvinyl | 20 |
| | polymer solution | |
| (9) | methyl parahydroxybenzoate | 0.1 |
| (10) | conagenin | 2 |
| (11) | potassium hydroxide | 0.1 |
| (12) | flavor | 0.2 |
| (13) | purified water | q.s. |

| | |
|---|---|
| total amount | 100 |

### Example 12

The formulation of skin cream is shown in Table 11. The oil phase components (1)-(7) in Table 11 were mixed, homogeneously dissolved and heated to 75° C. On the other hand, aqueous phase components (8), (9), (10) were mixed, dissolved and heated to 75° C. Then, the oil phase components were added to the above-mentioned aqueous phase components and preliminarily emulsified. The emulsion was uniformly emulsified in a homomixer. The mixture was cooled, (11) was added at 50° C. and mixed.

TABLE 11

| component | | amount added (wt %) |
|---|---|---|
| (1) | beeswax | 6 |
| (2) | cetanol | 5 |
| (3) | reduced lanolin | 8 |
| (4) | squalane | 37.5 |
| (5) | fatty acid glycerol | 4 |
| (6) | lipophilic glycerol monostearate | 2 |
| (7) | polyoxyethylene(20)sorbitan | 2 |
| | monolaurate | |
| (8) | propylene glycol | 5 |
| (9) | conagenin | 5 |
| (10) | methyl parahydroxybenzoate | 0.1 |
| (11) | flavor | 0.2 |
| (12) | purified water | q.s. |

| | |
|---|---|
| total amount | 100 |

### Example 13

The formulation of O/W type emulsion ointment type skin external preparation is shown in Table 12. The oil phase components (1)-(5) in Table 12 were mixed, homogeneously dissolved and heated to 75° C. On the other hand, aqueous phase components (5), (6), (7), (10) were mixed, dissolved and heated to 75° C. Then, the oil phase components were added to the above-mentioned aqueous phase components and emulsified. The mixture was cooled, (8), (9) were added at 50° C. and mixed.

TABLE 12

| component | | amount added (wt %) |
|---|---|---|
| (1) | white petrolatum | 25 |
| (2) | stearyl alcohol | 25 |
| (3) | glycerol | 12 |
| (4) | sodium lauryl sulfate | 1 |
| (5) | methyl parahydroxybenzoate | 0.025 |
| (6) | butyl parahydroxybenzoate | 0.025 |
| (7) | conagenin | 5 |
| (8) | allantoin | 1 |
| (9) | aloe extract | 1 |
| (10) | purified water | q.s. |

| | |
|---|---|
| total amount | 100 |

### Example 14

### Production of Whitening Cream

A: conagenin (1.00 g), purified water (5.00 g), B: 3-succinoyloxy disodium glycyrrhezinate (0.05 g), C: squalane (10.00 g), octyldodecyl myristate (8.00 g), microcrystalline wax (4.00 g), bechenyl alcohol (3.00 g), lipophilic glycerol monostearate (2.50 g), polyoxyethylene sorbitan monostearate (20 E.O., 2.50 g), D: 1,3-butylene glycol (10.00 g), methyl parahydroxybenzoate (0.10 g), purified water (54.00 g), and E: flavor (0.30 g)

[Production method] D was heated to 80-85° C., B was added, C dissolved by heating to 80-85° C. was added thereto while stirring in a homomixer, and they were uniformly emulsified. This was slowly cooled to about 50° C. at room temperature, and E and suspended A were added. The mixture was cooled to room temperature with stirring to give a whitening cream.

### Example 15

### Production of Carmine Lotion

A: zinc oxide (1.30 g), silicic anhydride (1.10 g), talc (2.00 g), red iron oxide (0.01 g), polyoxyethylenestearate amide (4 E.O., 0.05 g), B: conagenin of Example 1 (0.60 g), ethanol (5.00 g), purified water (5.00 g), C: conc. glycerol (3.00 g), camphor (0.10 g), methyl parahydroxybenzoate (0.05 g), flavor (0.05 g), and D: purified water (81.74 g)

[Production method] About 60 g of D was added to A, and they were uniformly dispersed in a homomixer to give a powder dispersion liquid. A solution of B, and C were added, the rest of D was added, and they were uniformly dispersed in a homomixer to give a carmine lotion.

### Example 16

### Production of Whitening Ointment

A: macrogol 4000 (47.50 g), macrogol 400 (47.50 g), and B: conagenin of Example 1 (0.50 g), purified water (4.50 g)

[Production method] Macrogol 4000 and macrogol 400 were dissolved by heating to 65° C. in a water bath, and uniformly mixed to give a macrogol ointment base. The base was kneaded with solution B to give a whitening ointment.

### INDUSTRIAL APPLICABILITY

According to the present invention, a safe and highly effective, fibroblast activator, collagen production promoter, collagen contraction promoter, hyaluronic acid production promoter, ATP production promoter or melanin formation suppressor can be provided. In addition, a safe skin external preparation, cosmetic (cosmetic composition), pharmaceutical product or food, which shows a high wrinkle improving effect can be provided. Furthermore, a safe skin external preparation, cosmetic (cosmetic composition), pharmaceutical product or food, which shows a high whitening effect can be provided.

This application is based on patent application Nos. 2005-212175, 2005-299047 and 2006-148622 filed in Japan, the contents of which are incorporated in full herein by this reference.

1. A skin external preparation or cosmetic containing at least one kind of compound selected from the group consisting of conagenin, a conagenin derivative, and a pharmaceutically acceptable salt thereof.

2. A fibroblast activator comprising, as an active ingredient, at least one kind of compound selected from the group consisting of conagenin, a conagenin derivative, and a pharmaceutically acceptable salt thereof.

3. A collagen production promoter comprising, as an active ingredient, at least one kind of compound selected from the group consisting of conagenin, a conagenin derivative, and a pharmaceutically acceptable salt thereof.

4. A collagen contraction promoter comprising, as an active ingredient, at least one kind of compound selected from the group consisting of conagenin, a conagenin derivative, and a pharmaceutically acceptable salt thereof.

5. A hyaluronic acid production promoter comprising, as an active ingredient, at least one kind of compound selected from the group consisting of conagenin, a conagenin derivative, and a pharmaceutically acceptable salt thereof.

6. An ATP production promoter comprising, as an active ingredient, at least one kind of compound selected from the group consisting of conagenin, a conagenin derivative, and a pharmaceutically acceptable salt thereof.

7. A melanin formation suppressor comprising, as an active ingredient, at least one kind of compound selected from the group consisting of conagenin, a conagenin derivative, and a pharmaceutically acceptable salt thereof.

8-10. (canceled)

11. A cosmetic comprising the activator, promoter or melanin formation suppressor of any one of claims 2 to 7.

12-15. (canceled)
